(19) United States
(12) Patent Application Publication
(10) Pub. No.: US 2007/0123974 A1
(43) Pub. Date: May 31, 2007

(54) Vascular stent which is specially designed for the multiple drug loading and better drug elution

(75) Inventors: Jeong Euy Park, Seoul (KR); Richard Sangwon Rhee, Anaheim Hills, CA (US)

Correspondence Address: FROMMER LAWRENCE & HAUG, 745 FIFTH AVENUE- 10TH FL., NEW YORK, NY, 10151, US

(73) Assignee: Samsung Life Welfare Foundation

(21) Appl. No.: 11/585,028

(22) Filed: Oct. 23, 2006

(30) Foreign Application Priority Data
Oct. 24, 2005 (KR) 10-2005-0100276

(51) Int. Cl. A61F 2/06

(52) U.S. Cl. 623/1.16

(57) ABSTRACT

Provided is a vascular stent used in percutaneous coronary intervention (PCI) which is specially designed for multiple drug loading and improved drug elution, including: a plurality of ring structures extending in the longitudinal direction of the vascular stent, including a plurality of struts disposed in a zigzag formation and connected to each other to form a cylindrical loop; and a plurality of link structures disposed between the ring structures and connecting the ring structures in the longitudinal direction of the vascular stent, wherein each of the struts in a ring structure is connected to adjacent struts in the same ring structure through one of a plurality of linking ends, a slot loaded with drugs is formed in the strut in the longitudinal direction of the strut, and a multi-layer structure comprising a plurality of layers of drugs is loaded in the slot. The vascular stent effectively inhibits restenosis by loading a large amount of a drug or various types of drugs in multiple layers in slots of struts in the vascular stent and controlling elution of the drugs, and can be easily installed in a serpentine coronary artery with excellent flexibility. In addition, the link structure of the vascular stent is disposed in a symmetric formation, and thus, side branch accessibility is excellent and an additional coronary intervention can be performed easily in a branched artery.

### CROSS-REFERENCE TO RELATED PATENT APPLICATION

This application claims the benefit of Korean Patent Application No. 10-2005-0100276, filed on Oct. 24, 2005, in the Korean Intellectual Property Office, the disclosure of which is incorporated herein in its entirety by reference.

### BACKGROUND OF THE INVENTION

1. Field of the Invention

The present invention relates to a vascular stent which is specially designed for multiple drug loading and improved drug elution, and more particularly to a vascular stent used in percutaneous coronary intervention (PCI), which effectively inhibits restenosis by loading a large amount of a drug or various types of drugs in multiple layers in slots of struts in the vascular stent and controlling elution of the drugs from the vascular stent to blood vessels, and can be easily installed in a serpentine coronary artery with excellent flexibility.

2. Description of the Related Art

Percutaneous coronary intervention (PCI) is a treatment procedure for obstructive coronary artery diseases such as myocardial infarction and angina pectoris. The procedure involves dilating the narrowed coronary artery by inserting a guidewire and then a balloon catheter into the obstructed coronary artery segment through arteries in the wrists or groins and expanding a balloon to expand the narrowed lesion. PCI is widely known as the most effective way to treat obstructed coronary arteries. It is estimated that more than 1 million patients in U.S.A., more than 100,000 patients in Japan, and more than 15,000 patients in Korea undergo PCI each year.

In PCI, the narrowed coronary arterial wall is expanded using the balloon catheter. In over 70% of patients undergoing PCI, a stent, which is a thin stainless steel or cobalt chrome mesh tube, is inserted in a vascular wall and thus the expanded vascular wall is continuously sustained.

FIGS. 1A through 1D are schematic cross sectional views of blood vessels illustrating a PCI treatment process using a conventional stent and a restenosis formation;

Referring to FIGS. 1A through 1D, a PCI treatment process using a balloon catheter with a stent will be described.

First, a balloon catheter 2 including a balloon 2a and a stent 1a optimized in conditions such as the length of a stenotic lesion and the diameter of a blood vessel, etc. is selected and inserted into a stenotic lesion L of a coronary artery (CA) (FIG. 1A). When the balloon catheter 2 reaches an accurate location of the stenosis region L, the balloon 2a is expanded to expand the stent 1a which is mounted on the balloon 2a, so that the stent 1 is expanded by plastic deformation(FIG. 1B). Then, the expanded balloon 2a is deflated to remove the balloon catheter 2 including the balloon 2a, and thus the stent la is left installed in the coronary artery to keep the vascular region L open and prevent the coronary artery from being renarrowed (FIG. 1C). However, since the stent 1a itself is a foreign material in the human body, the cells in the vascular wall which withstand pressure from the installed stent 1a sustain barotrauma, and thus rapidly proliferate. If the rapidly proliferated cells excessively cover the stent 1a, the vascular wall is narrowed again resulting in restenosis L′ (FIG. 1D). The risk of restenosis increases as the length of the stent 1a increases and as the diameter of the stent 1a decreases, and restenosis occurs in approximately 17 to 25% of patients. Restenosis mainly occurs within 1 to 3 months after PCI, and rarely occurs after 6 months.

Restenosis has been one of the major limitations of PCI. Accordingly, various methods of preventing restenosis have been developed. Recently, stents coated with drugs which can inhibit tissue overgrowth on the stent and prevent restenosis have been widely used in PCI treatment, resulting in a remarkable decrease in the incidence of restenosis after PCI.

Drugs such as rapamycin or paclitaxel which have anti-cancer activities are coated on the stents to inhibit restenosis after PCI treatment. When the drug coated stents are installed in the coronary artery, the restenosis rate is about 4% at a proximal end P and about 2 to 3% at a distal end D. It is assumed that the restenosis rate is relatively higher at the proximal end P than at the distal end D since the drugs coated on the stents to inhibit restenosis are washed by the bloodstream from the proximal end P to the distal end D.

Stents coated with drugs which can prevent restenosis are expected to become more widely used in coronary intervention in the future. Stents coated with drugs which can prevent restenosis have already been widely used. However most of the currently used drug coated stents use their preexisting stents and are not specifically designed for drug coating purpose. Stent design also has limitations that some stents are rigid to conform and track to the tortuous vessel and in side branch accessibility.

Thus, newly-designed stents for loading a sufficient amount of a drug or drug combinations which can sustainedly elute the drugs for a long period of time are required.

One of the inventors of the present invention recognized that conventional stents should be modified and filed a patent application (Korean Patent Application No. 2003-3465) reciting a stent for PCI which can be coated with a vascular restenosis-preventing drug with the Korean Intellectual Property Office. The structure of the stent is illustrated in FIGS. 2A and 2B.

FIG. 2A is a perspective view of an expanded stent 1b for PCI according to another invention of the inventor of the present invention, and FIG. 2B is an open form of the stent 1b. The stent 1b is formed by disposing several first ring structures 10 and several second ring structures 20 in the longitudinal direction of the stent 1b. Each of the first ring structures 10 includes a plurality of struts 11 disposed in a zigzag formation and connected to each other to form a cylindrical loop. A hole filled with drugs 12a penetrates each of a plurality of round ends 12 connecting each of the struts 11 and faces the center of the stent 1b. A groove filled with drugs 11a is formed on the surface of each of the struts 11 in the longitudinal direction of the struts 11.

Meanwhile, each of the second ring structures 20 includes a plurality of struts 21 having a thread 22 and a chase 23 disposed in a zigzag formation and connected to each other to form a cylindrical loop. The second ring structure 20 also includes a plurality of bridges 30 connecting one point of the struts 11 of the first ring structure 10 and the threads 22 of the second ring structure 20. The bridges 30 are used to connect the first ring structures 10 and the second structures 20 to each other to form a net, and the bridges 30 includes a N-type serpentine link 31 in the center thereof.

The stent 1b illustrated in FIGS. 2A and 2B, which was invented by one of the inventors of the present invention, has an excellent effect on eluting drugs inhibiting restenosis by loading the drugs on the stent 1b. However, a vascular stent needs to be newly designed for loading a large amount of drugs inhibiting restenosis which occurs after PCI, extending the elution time of the drugs by separating a plurality of drugs in multiple layers, and controlling the elution time.

In addition, a vascular stent needs to be newly designed to be inserted into and installed in a serpentine coronary artery with excellent flexibility and to have open cell structures through which another balloon catheter with a stent can be inserted into and installed in a branched artery.

### SUMMARY OF THE INVENTION

The present invention provides a specially designed vascular stent for multiple drug loading and improved drug elution which effectively elutes drugs for a long period of time by loading a large amount of a drug or various types of drugs in multiple layers in slots of struts in the vascular stent.

The present invention also provides an open cell type vascular stent with excellent flexibility having an open gap through which a balloon catheter with a stent can be inserted.

According to an aspect of the present invention, there is provided a vascular stent which is specially designed for multiple drug loading and better drug elution including:

a plurality of ring structures extending in the longitudinal direction of the vascular stent, comprising a plurality of struts disposed in a zigzag formation and connected to each other to form a cylindrical loop; and

a plurality of link structures disposed between the ring structures and connecting the ring structures in the longitudinal direction of the vascular stent,

wherein each of the struts in a ring structure is connected to adjacent struts in the same ring structure through one of a plurality of linking ends, a slot is formed in the strut in the longitudinal direction of the strut, and a multi-layer structure comprising a plurality of layers of drugs is loaded in the slot.

### BRIEF DESCRIPTION OF THE DRAWINGS

The above and other features and advantages of the present invention will become more apparent by describing in detail exemplary embodiments thereof with reference to the attached drawings in which:

FIGS. 1A through 1D are schematic cross sectional views of blood vessel illustrating a percutaneous coronary intervention (PCI) treatment process using a conventional stent and a restenosis formation;

FIG. 2A is a perspective view of an expanded stent 1b for PCI according to another invention of the inventor of the present invention;

FIG. 2B is an open form view of the stent 1b illustrated in FIG. 2A;

FIG. 3 is a perspective view of a vascular stent 1 for multiple drug loading and improved drug elution according to an embodiment of the present invention;

FIG. 4 is an open form view of the vascular stent 1 of FIG. 3, according to an embodiment of the present invention;

FIG. 5 is an open form view of the vascular stent 1 of FIG. 3 when expanded, according to an embodiment of the present invention;

FIG. 6 is an enlarged view of a section of the vascular stent 1 of FIG. 4, according to an embodiment of the present invention;

FIGS. 7A and 7B are cross sectional views of a strut 120 in the enlarged vascular stent 1 of FIG. 6 taken along an arbitrarily drawn cut-line VII-VII of FIG. 6, illustrating a rectangular slot 121 loaded with one drug, and a rectangular slot 121 loaded with a plurality of layers of drugs, respectively, according to an embodiment of the present invention;

FIG. 8A schematically illustrates a vascular stent 1 in which a rectangular slot 121 is loaded with one drug and the external surface of the vascular stent 1 is coated with another drug, according to an embodiment of the present invention;

FIG. 8B is a graph illustrating the concentration of drugs eluted from the vascular stent of FIG. 8A according to time, according to an embodiment of the present invention;

FIG. 9A schematically illustrates a vascular stent 1 in which a rectangular slot 121 is loaded with a plurality of layers of drugs and the external surface of the vascular stent 1 is coated with another drug, according to an embodiment of the present invention;

FIG. 9B is a graph illustrating the concentration of coated drugs eluted from the stent of FIG. 9A according to time, according to an embodiment of the present invention;

FIG. 10 is an enlarged perspective view of a partial stent 1 in which an open gap 210 is formed between S-type links 200′ of a link structure 200, according to an embodiment of the present invention;

FIGS. 11A through 11D illustrate procedures of inserting a balloon catheter 2 with a second vascular stent 1″ into a branched artery BA through an open gap 210 formed between S-type links 200′ of a first vascular stent 1′ installed in a coronary artery CA, and installing the second vascular stent 1″ in the branched artery BA when stenosis regions L1 and L2 are found in each of the coronary artery CA and the branched artery BA, according to an embodiment of the present invention;

FIG. 12 is a schematic view of a stent 1 installed in a serpentine blood vessel, according to an embodiment of the present invention;

FIGS. 13A and 13B illustrate sizes of elements of a stent 1 according to an embodiment of the present invention;

FIG. 14 illustrates an open form of a stent 1 according to another embodiment of the present invention;

FIG. 15 illustrates an open form of a stent 1 according to another embodiment of the present invention; and

FIG. 16 illustrates an open form of a stent 1 according to another embodiment of the present invention.

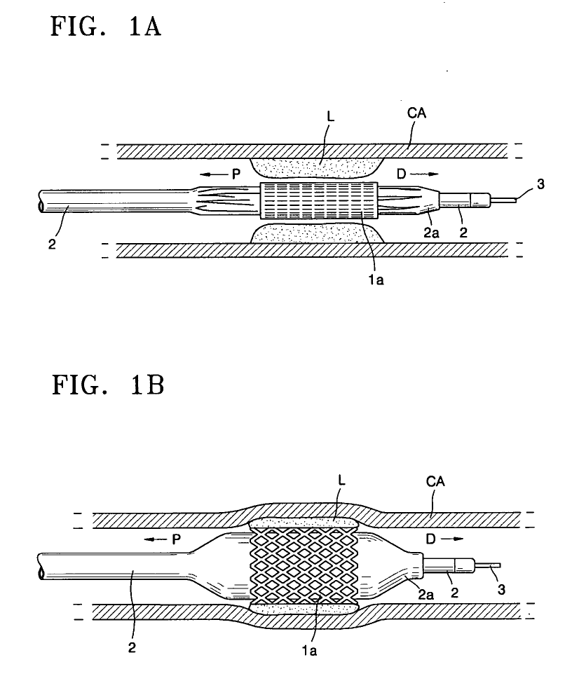

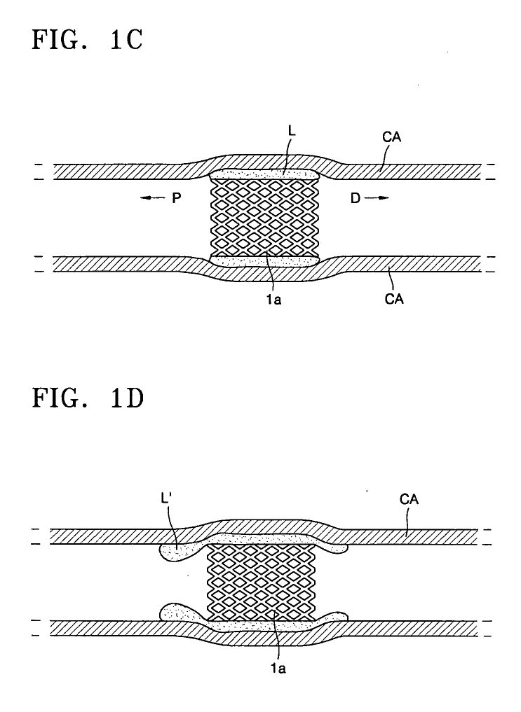

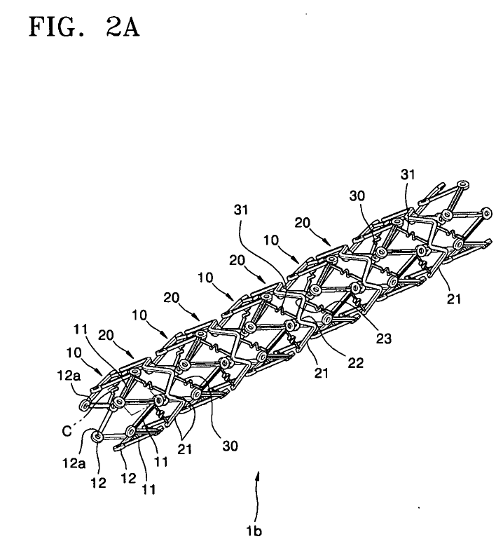

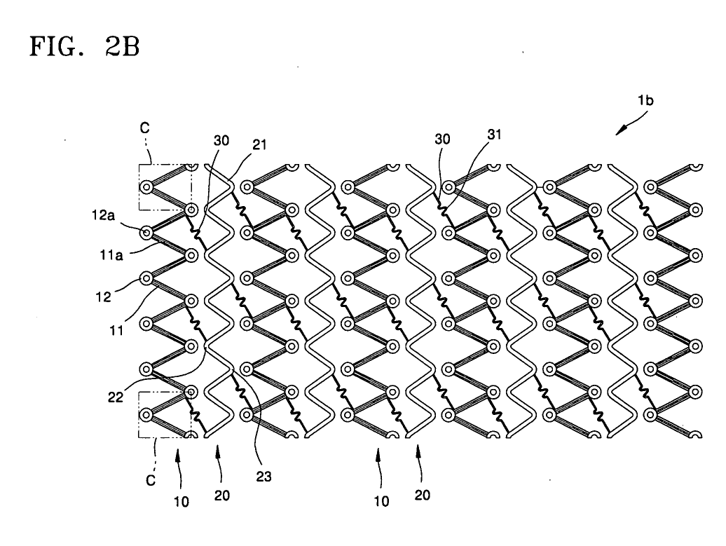

### DETAILED DESCRIPTION OF THE INVENTION

Hereinafter, the structure and effect of a vascular stent of the present invention which is specially designed for multiple drug loading and improved drug elution will be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art.

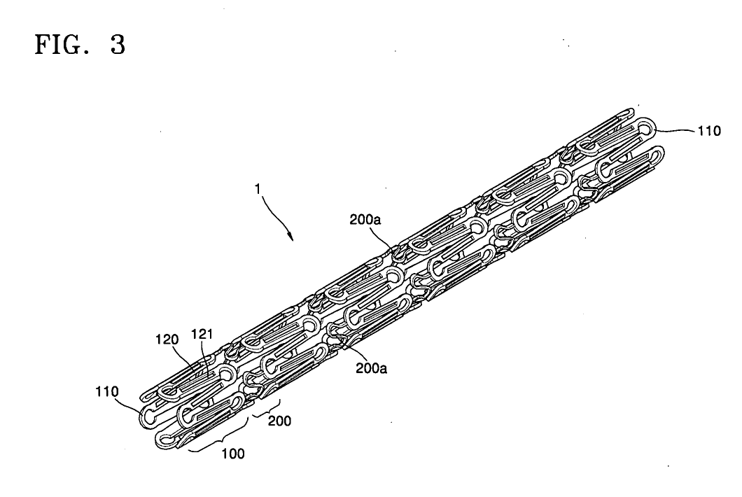

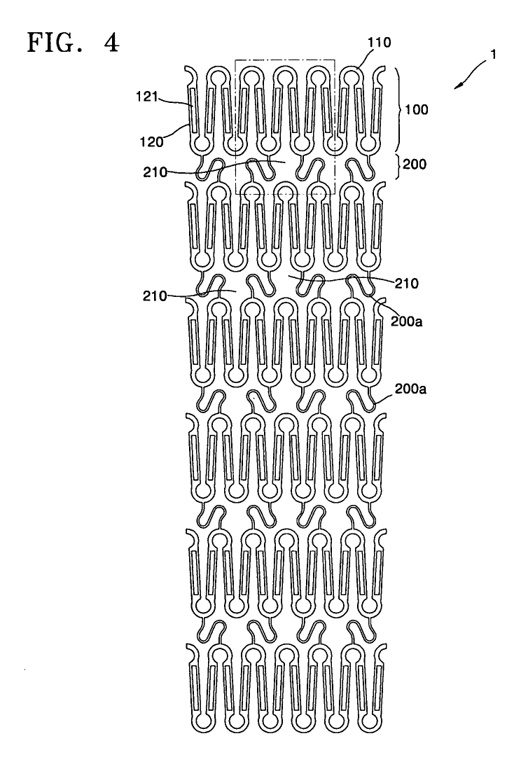

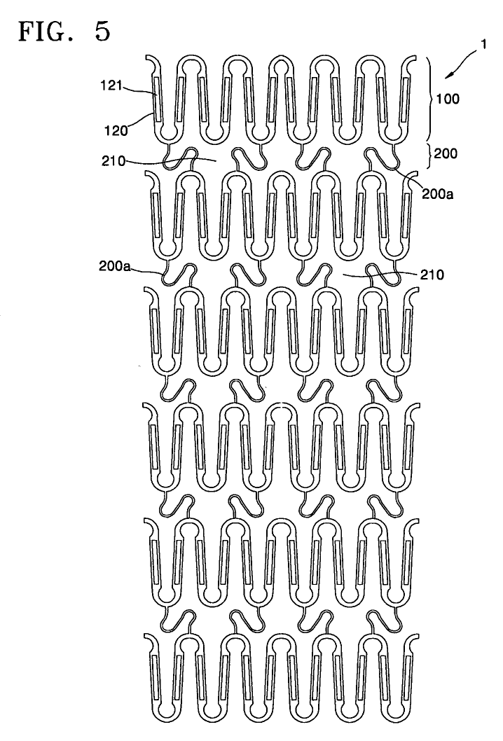

FIG. 3 is a perspective view of a vascular stent 1 which is specially designed for multiple drug loading and improved drug elution according to an embodiment of the present invention. FIG. 4 is an open form view of the vascular stent 1 of FIG. 3, and FIG. 5 is an open form view of the vascular stent 1 of FIG. 3 when expanded;

Referring to FIGS. 3 through 5, the stent 1 according to the current embodiment of the present invention includes ring structures 100 in which struts 120 are connected to each other by linking ends 110 in a zigzag formation, and link structures 200 including S-type links 200a connecting the ring structures 100. In the ring structures 100, rectangular slots 121 loaded with drugs are formed in the struts 120 in the longitudinal direction of the struts 120 and penetrating the struts 120. One end of each of the struts 120 is connected to a corresponding one of the linking ends 110 having an open ring shape.

In the link structures 200 connecting the ring structures 100 of the stent 1, each of both ends of each of the S-type links 200a is connected to a corresponding one of the linking ends 110 of a first ring structure 100 and a corresponding one of the linking ends 110 of a second ring structure 100. The thickness of the S-type links 200a is less than the width of the struts 120 to facilitate bending and expansion of the vascular stent 1. Due to such property, the vascular stent according to the current embodiment of the present invention can be installed in a serpentine blood vessel.

The amount of ring structures 100 and link structures 200 may vary according to the length of the vascular stent 1. The vascular stent 1 typically has 6 to 8 ring structures 100, and ring structures 100 should be disposed at both ends of the vascular stent. The vascular stent 1 illustrated in FIG. 3 consists of 6 ring structures 100 and 5 link structures 200.

When the struts 120 get twisted or bent during the expansion of the struts 120, drugs may leak out of the rectangular slots 121 loaded with drugs. Referring to FIGS. 3 and 4, the thickness of the linking ends 110 may be less than that of the struts 120 in the ring structures 100 in order to facilitate expansion of the vascular stent 1 by easily spreading both ends of the linking ends 110 during the expansion of a balloon and not to deform the struts 120.

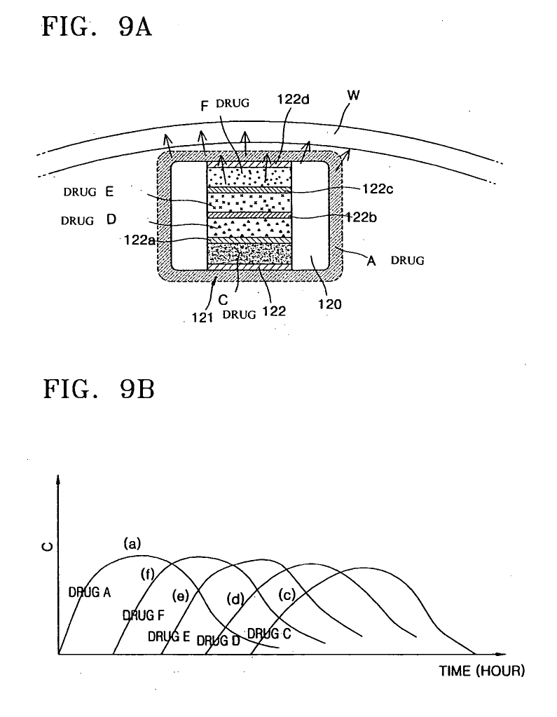

In the vascular stent 1 according to the current embodiment of the present invention, the rectangular slots 121 loaded with drugs are formed in the struts 120 in the longitudinal direction of the struts 120 and penetrating the struts 120 to contain a larger amount of drugs than a conventional stent. Only one drug may be loaded in the rectangular slots 121, or a plurality of layers of drugs may be loaded in the rectangular slots 121 (FIG. 7B and 9A). The size of the rectangular slots 121 loaded with drugs may vary according to the size and length of the vascular stent 1.

Generally, the rectangular slots 121 loaded with drugs may have an area of from 0.05 inch (1.27 mm)×0.002 inch (0.05 mm) to 0.20 inch (5.0 mm)×0.008 inch (0.2 mm).

The amount of struts 120 to be included in one ring structure 100 may be determined in consideration of the diameter of the vascular stent 1 when the vascular stent 1 is expanded. That is, if the diameter of the vascular stent 1 is about 3.0 to 3.5 mm when the vascular stent 1 is expanded, each of the ring. structures 100 of the vascular stent 1 may have 12 to 14 struts 120 since it is preferable to have 12 to 14 rectangular slots 121 loaded with drugs. When the diameter of the vascular stent 1 is greater than about 3.5 mm, the amount of struts 120 is required to be increased.

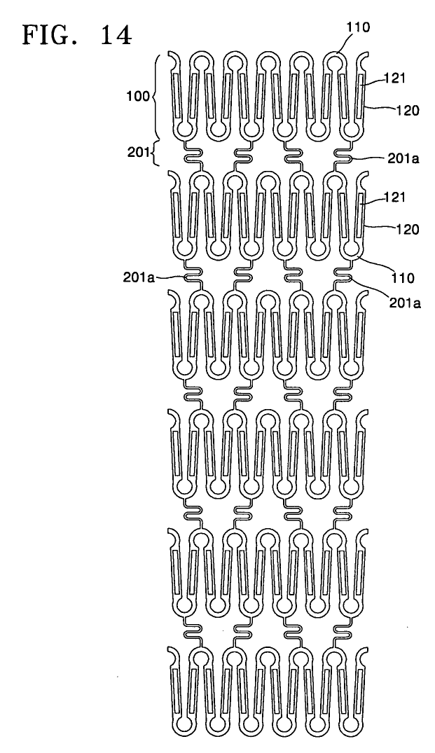

Referring to FIGS. 3 through 5, the S-type links 200a in the link structures 200 of the vascular stent 1 may have a width of about 0.05 mm in order not to cause any difficulty during the installation of the vascular stent 1 in a blood vessel. In FIGS. 3 through 5, the S-type links 200a are illustrated. However, other types of links such as “N”-type links, “V”-type links and “W”-type links can be used as illustrated in FIGS. 14 through 16.

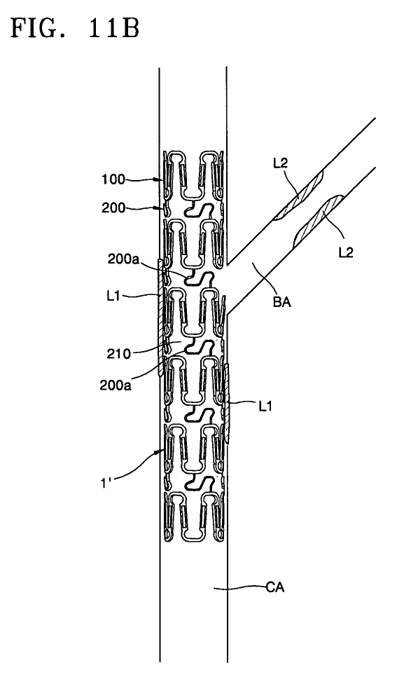

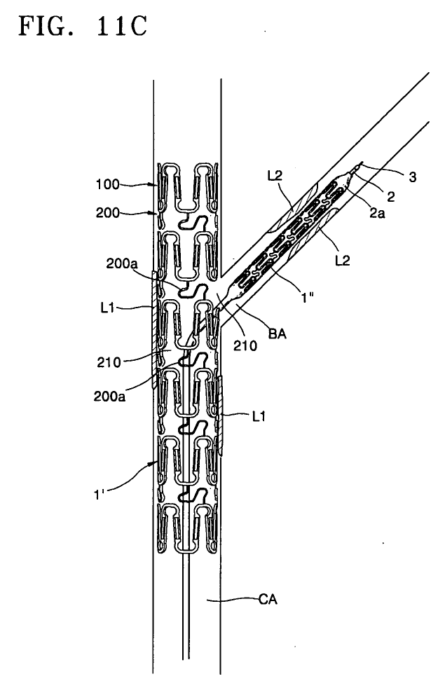

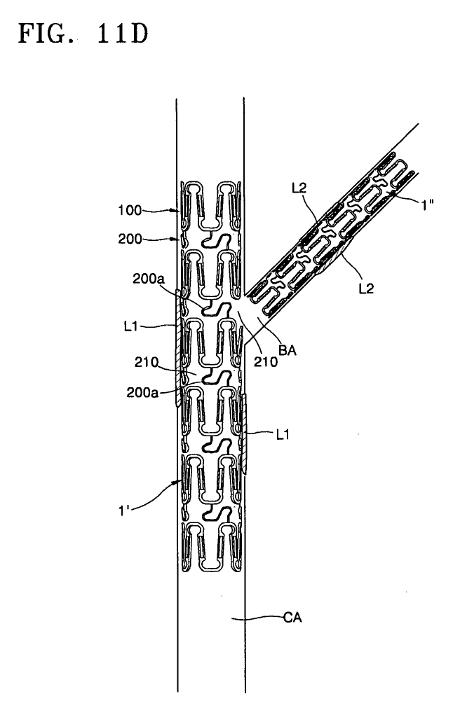

In addition, all of the links 200a in the link structures 200 of the vascular stent 1 are not disposed in the same direction. The links 200a are disposed in asymmetric formation in which a couple of links 200a are symmetric to each other to form an open gap as large as possible between the links 200a. When stenosis regions are found in a coronary artery CA and a branched artery BA, a first stent may be installed in the coronary artery CA and a second stent with a balloon catheter may be easily inserted through the obtained large open gap between the links and installed in the branched artery BA as illustrated in FIGS. 11A through 11D.

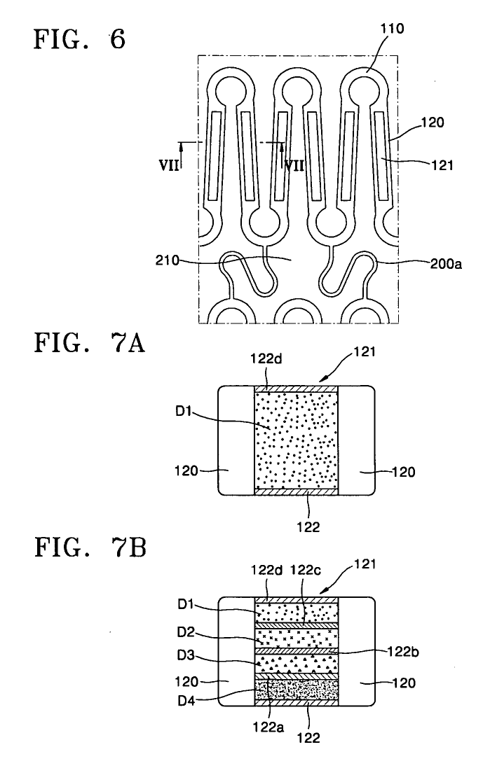

FIG. 6 is an enlarged open form view of part of the vascular stent 1 of FIG. 4. FIGS. 7A and 7B are cross sectional views of one of the struts 120 in the vascular stent 1 taken along an arbitrarily drawn cut-line VII-VII of FIG. 6, illustrating a rectangular slot 121 loaded with one drug, and a rectangular slot 121 loaded with a plurality of layers of drugs, respectively, according to an embodiment of the present invention.

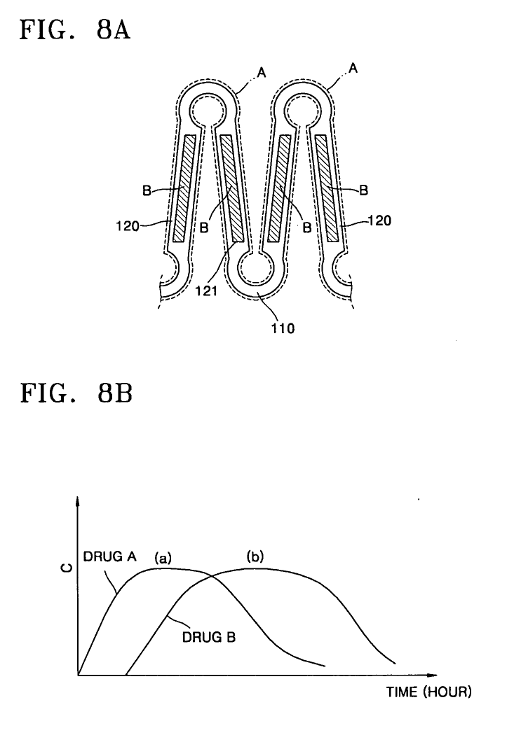

In FIG. 7A, the top of the rectangular slot 121 is in contact with an inner vascular wall and the bottom of the rectangular slot 121 is disposed toward the inside of a blood vessel. A base layer 122 is formed at the bottom of the rectangular slot 121 to prevent drugs from leaking into the blood vessel. A drug 1 (D1) is loaded onto the base layer 122, and an isolation layer 122d is formed at the top of the rectangular slot 121, that is, the isolation layer 122d is in contact with the inner vascular wall. As illustrated not in FIGS. 6 and 7A, but in FIGS. 8A and 9A, the external surface of the vascular stent 1 may be coated with another drug, and thus the isolation layer 122d should be formed on the top of the rectangular slot 121 to prevent interference by the coated drug.

FIG. 7B illustrates a rectangular slot 121 loaded with a plurality of layers of drugs. In FIG. 7B, a base layer 122 is formed on the bottom of the rectangular slot 121 of the strut 120. The base layer 122 is disposed toward the inside of a blood vessel, and a drug 4 (D4), an isolation layer 122a, a drug 3 (D3), an isolation layer 122b, a drug 2 (D2), an isolation layer 122c, a drug 1 (D1), and an isolation layer 122d are sequentially loaded on the base layer 122. When a plurality of drugs are loaded in a multi-layer structure, the drugs may be sequentially eluted. Thus, drug elution time can be controlled, and the effect of the drugs can be sustained for a long period of time.

FIG. 8A schematically illustrates a vascular stent 1 in which a rectangular slot 121 is loaded with one drug B and the external surface of the vascular stent 1 is coated with another drug A, according to an embodiment of the present invention. FIG. 8B is a graph illustrating the concentration of drugs eluted from the vascular stent 1 of FIG. 8A according to time. In the vascular stent 1 of FIG. 8A, since drug A which is coated on the external surface of the vascular stent 1 is eluted and then drug B is eluted, the concentration of drug A (curve (a)) is high at the initial stage and the concentration of drug B (curve (b)) increases while the concentration of drug A decreases as illustrated in FIG. 8B.

FIG. 9A schematically illustrates a vascular stent 1 in which a rectangular slot 121 is loaded with a plurality of layers of drugs (drugs C, D, E, and F) and the external surface of the vascular stent 1 is coated with another drug A, according to an embodiment of the present invention. FIG. 9B is a graph illustrating the concentration of coated drugs eluted from the vascular stent 1 of FIG. 9A according to time. According to FIG. 9B, the concentration of drug A (curve (a)) is high at the initial stage and the drug elution effects of drug F, drug E, drug D, and drug C are sequentially obtained (curves (f), (e), (d), and (c))

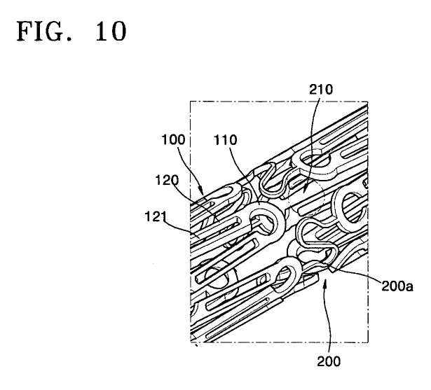

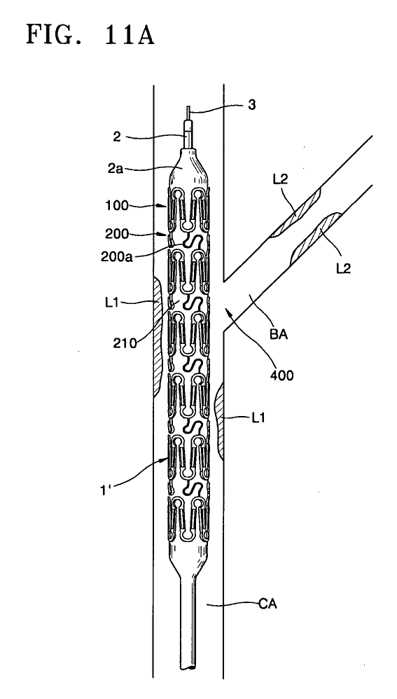

FIG. 10 is an enlarged perspective view of a portion of a vascular stent 1 in which an open gap 210 is formed between S-type links 200a of a link structure 200, according to an embodiment of the present invention

FIGS. 11A through 11D illustrate procedures of inserting a balloon catheter 2 with a vascular stent 1″ into a branched artery BA through an open gap 210 formed between S-type links 200a of a vascular stent 1′ installed in a coronary artery CA, and installing the vascular stent 1″ in the branched artery BA when stenosis regions L1 and L2 are found in each of the coronary artery CA and the branched artery BA.

As illustrated in FIG. 11A, more than one stenosis region may be found in the vicinity of a branch point 400 in the coronary artery CA. In such case, the balloon catheter 2 including a balloon 2a with a vascular stent 1′ is inserted into the coronary artery CA to treat a first stenosis region L1 as illustrated in FIG. 11A, and the balloon 2a is expanded to install the vascular stent 1′ as illustrated in FIG.11B. Then, another balloon catheter 2 including a balloon 2a with a vascular stent 1″ is inserted into the branched artery BA through an open gap between the S-type links 200a of a link structure 200 of the vascular stent 1′ to treat a second stenosis region L2 as illustrated in FIG. 11C, and the balloon 2a is expanded to install the vascular stent 1″ as illustrated in FIG. 11D.

The stent of an embodiment of the present invention includes the link structure 200 arranged in an open cell type and the links 200a which is disposed in a mirror-symmetric manner in which a couple of links 200a are facing each other to form an open gap 210 between the links 200a larger than that of a conventional stent.

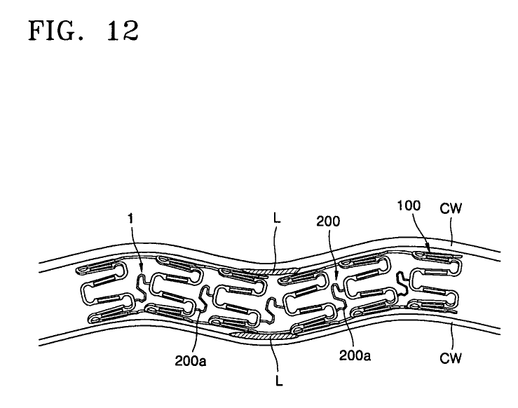

FIG. 12 is a schematic view of a vascular stent 1 which is installed in a serpentine blood vessel, according to an embodiment of the present invention. The vascular stent 1 may have excellent flexibility since links 200a of the vascular stent 1 are thin. Thus, the vascular stent 1 can be easily installed in the serpentine blood vessel.

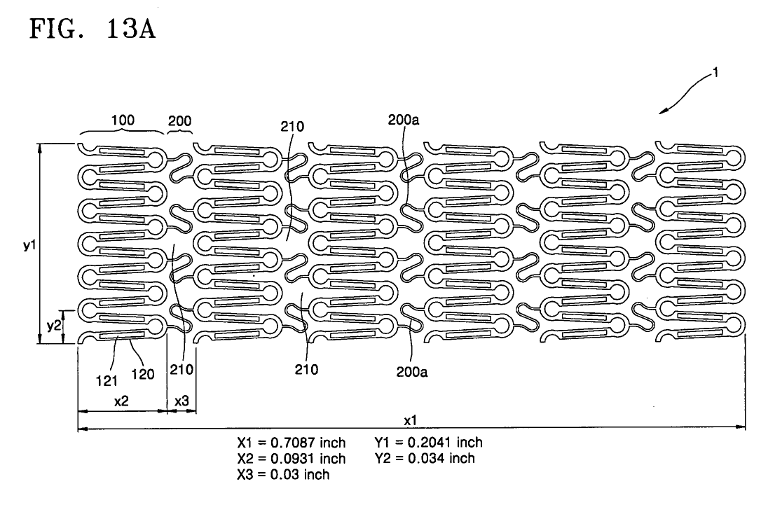

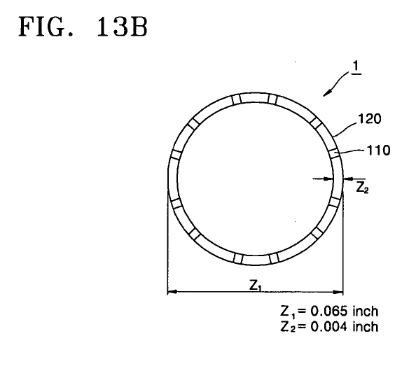

FIGS. 13A and 13B illustrate details of a vascular stent 1 according to an embodiment of the present invention. A material that is used to form the vascular stent 1 may be stainless steel or cobalt-chrome, and a material in a cylindrical shape may be laser-cut to form the vascular stent 1.

According to an embodiment of the present invention, the entire length Xl of the vascular stent 1 including 6 ring structures 100 and 5 link structures 200 may be 0.7087 inch. The length X2 of each of the ring structures 100 may be 0.0931 inch, and the width Y1 of each of the ring structures 100 when not expanded may be 0.2041 inch. In addition, the distance Y2 between linking ends 110 may be 0.0340 inch, and the length X3 of each of the link structures 200 may be 0.03 inch (FIG. 13A).

Meanwhile, referring to FIG. 13B, the external diameter Z1 of the vascular stent 1 when not expanded may be 1.65 mm (0.065 inch), and the thickness Z2 of the strut 120 may be 0.004 inch.

The sizes of each part of the vascular stent 1 are not limited thereto and may vary according to various conditions and purposes for which the vascular stent 1 are to be used. 90% of stent products that are commonly used in the art are made of a cobalt-chrome alloy, since cobalt-chrome stents are effective for preventing restenosis in blood vessels without drug coatings, and have excellent corrosion resistance and long fatigue life compared to conventionally-used stainless steel stents. Thus, cobalt-chrome stents can have advantages compared with stainless steel stents.

A method of manufacturing a vascular stent according to an embodiment of the present invention may include the following processes. A stainless steel or cobalt-chrome material having a cylindrical shape is laser-cut, in which metal is burned to be removed, to form a vascular stent. Then, the rough surface of the vascular stent is polished using a polishing process such as a chemical etching process to form a smooth surface.

Since the surface of the s vascular tent is directly in contact with vascular walls in the human body, the surface of the vascular stent need to be as smooth as possible and accordingly the polishing process affects the quality of the vascular stent. In an experiment, a laser-cut stent was polished three times, five times and eight times, and the results were compared with each other. While the surface of the vascular stent became smoother as the number of polishing processes increased, the manufacturing costs for the vascular stent increased, and the thickness and width of the metal parts of the vascular stent decreased. Accordingly, the optimum amount of polishing processes may be determined to achieve the desired manufacturing costs and quality of the vascular stent, and the size of the material to be used to form the vascular stent needs to be sufficient in consideration of the amount removed during the polishing process.

Then, drugs are loaded in a slot of the vascular stent and coated on the external surface of the vascular stent. Drugs which can prevent the proliferation of smooth muscle cells and prevent restenosis, such as rapamycin, paclitaxel or newer drugs having this property may be used. When the drugs which can prevent the proliferation of smooth muscle cells (and also may enhance reendothelization) are delivered to a damaged vascular wall due to a coronary intervention, the drugs inhibit the cell cycle regulators of proliferation in vascular cells, and thus restenosis can be prevented. In addition, inflammation inhibitors such as dexamethasone, gene therapy products, and the like can be used as restenosis preventing drugs, and estrogen based drugs containing the female hormone may also be used. A drug which inactivates metalloproteinase which is involved in collagenous fiber generation during cell proliferation may also be used. Any restenosis inhibiting drug may be loaded as much as possible in the vascular stent according to an embodiment of the present invention, so that the drug may be sustainedly eluted over a long period of time. In the specially designed stent of the present invention, conventional drugs may be loaded, and any drug that is to be developed in the future may also be loaded.

Meanwhile, a spray or dipping method or other methods may be used to coat the surface of the vascular stent. In the spray method, surface tension of the drug increases, and thus loading the drugs in the slot, which may be narrow, may be difficult. Any method that has been used or is to be developed, to coat drugs may be applied to the vascular stent manufacturing process.

In another embodiment of the present invention, “N”-type links, “V”-type links and “W”-type links may be used in addition to S-type links.

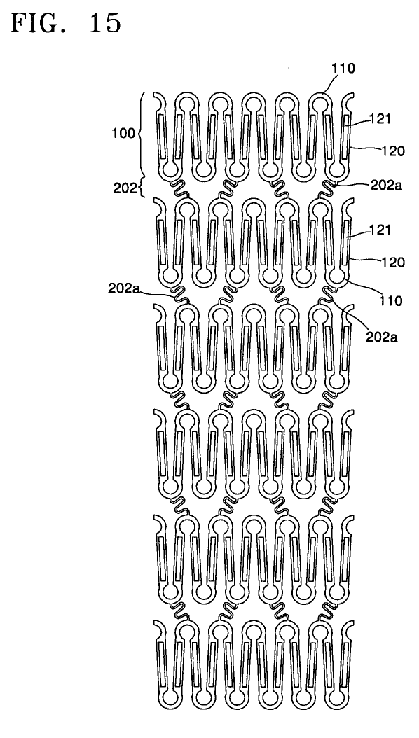

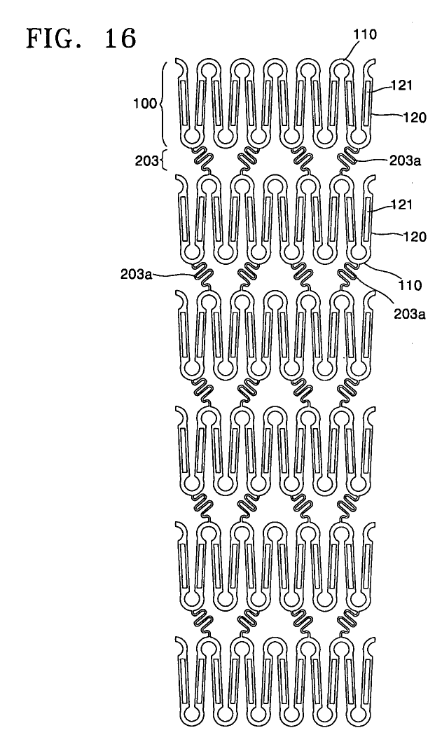

FIG. 14 illustrates an open form of a vascular stent 1 having an N-type link structure 201, according to another embodiment of the present invention. FIG. 15 illustrates an open form of a vascular stent 1 having a V-type link structure 202, according to another embodiment of the present invention and FIG. 16 illustrates an open form of a stent 1 having a W-type link structure 203, according to another embodiment of the present invention.

The stents having such link structures according to embodiments of the present invention can be easily expanded, and flexibly bent, and thus can be easily and safely installed in blood vessels.

As described above, a vascular stent according to the present invention which is specially designed for multiple drug loading and improved drug elution, effectively elutes drugs inhibiting restenosis for a long period of time by loading a large amount of a drug or various types of drugs in multiple layers in rectangular through-hole slots in struts in the vascular stent.

In addition, the vascular stent according to the present invention has a plurality of link structures and a plurality of linking ends having improved flexibility, and thus the vascular stent can be easily installed even in serpentine blood vessels.

The link structures of the vascular stent according to the present invention are disposed in a symmetric structure to form a larger open gap compared to a conventional stent, and thus an additional coronary intervention may be performed in a branched artery without a stent jail when the vascular stent is installed at a branch point of a coronary artery.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A vascular stent used in percutaneous coronary intervention (PCI), which is specially designed for multiple drug loading and better drug elution comprising:
a plurality of ring structures extending in the longitudinal direction of the vascular stent, comprising a plurality of struts disposed in a zigzag formation and connected to each other to form a cylindrical loop; and
a plurality of link structures disposed between the ring structures and connecting the ring structures in the longitudinal direction of the vascular stent,
wherein each of the struts in a ring structure is connected to adjacent struts in the same ring structure through one of a plurality of linking ends, a slot is formed in the strut in the longitudinal direction of the strut, and a multi-layer structure comprising a plurality of layers of drugs is loaded in the slot.

2. The vascular stent of claim 1, wherein the thickness of the linking ends is less than the thickness of the struts.

3. The vascular stent of claim 1, wherein each of the link structures comprises a plurality of S-type links, wherein a first end of each of the S-type links is connected to a linking end of the plurality of linking ends of a first ring structure of the plurality of ring structures disposed closer to a distal end of the vascular stent than a second ring structure of the plurality of ring structures and a second end of each of the S-type links is connected to a linking end of the plurality of linking ends of the second ring structure disposed closer to a proximal end of the vascular stent than the first ring structure.

4. The vascular stent of claim 3, wherein each of the link structures comprises an open gap between adjacent S-type links which extend in a circumferential direction of the vascular stent and are symmetrical to each other.

5. The vascular stent of claim 1, wherein each of the link structures comprises a plurality of N-type links, wherein a first end of each of the N-type links is connected to a linking end of the plurality of linking ends of a first ring structure of the plurality of ring structures disposed closer to a distal end of the vascular stent than a second ring structure of the plurality of ring structures and a second end of each of the N-type links is connected to a linking end of the plurality of linking ends of the second ring structure disposed closer to a proximal end of the vascular stent than the first ring structure.

6. The vascular stent of claim 5, wherein each of the link structures comprises an open gap between adjacent N-type links which extend in a circumferential direction of the vascular stent and are symmetrical to each other.

7. The vascular stent of claim 1, wherein each of the link structures comprises a plurality of V-type links, wherein a first end of each of the V-type links is connected to a linking end of the plurality of linking ends of a first ring structure of the plurality of ring structures disposed closer to a distal end of the vascular stent than a second ring structure of the plurality of ring structures and a second end of each of the V-type links is connected to a linking end of the plurality of linking ends of the second ring structure disposed closer to a proximal end of the vascular stent than the first ring structure.

8. The vascular stent of claim 7, wherein each of the link structures comprises an open gap between adjacent V-type links which extend in a circumferential direction of the vascular stent and are symmetrical to each other.

9. The vascular stent of claim 1, wherein each of the link structures comprises a plurality of W-type links, wherein a first end of each of the W-type links is connected to a linking end of the plurality of linking ends of a first ring structure of the plurality of ring structures disposed closer to a distal end of the vascular stent than a second ring structure of the plurality of ring structures and a second end of each of the W-type links is connected to a linking end of the plurality of linking ends of the second ring structure disposed closer to a proximal end of the vascular stent than the first ring structure.

10. The vascular stent of claim 9, wherein the link structure comprises an open gap between adjacent W-type links which extend in a circumferential direction of the vascular stent and are symmetrical to each other.
